(19) United States
(12) Patent Application Publication
(10) Pub. No.: US 2026/0014117 A1
(43) Pub. Date: Jan. 15, 2026

(54) ENHANCED ARTEMISININ-BASED COMBINATION THERAPY FOR TREATING PARASITIC MEDIATED DISEASE

(71) Applicant: KRYPTONITE GROUP, LTD., Freeport (BS)

(72) Inventors: Robert Lewis Steele, Palm Beach Gardens, FL (US); Anthony Fedele Musso, West Palm Beach, FL (US); David D. Mundschenk, Hollywood, FL (US); Russell Van de Casteele, Pompano Beach, FL (US)

(21) Appl. No.: 19/331,085

(22) Filed: Sep. 17, 2025

Related U.S. Application Data
(63) Continuation of application No. 14/201,740, filed on Mar. 7, 2014, now abandoned.
Application No. 14/201,740 is a continuation-in-part of application No. 13/542,719, filed on Jul. 6, 2012, now abandoned.
(60) Provisional application No. 61/774,664, filed on Mar. 8, 2013.

(51) Int. Cl. A61K 31/357; A61K 31/365; A61K 31/4706; A61K 31/4741

(52) U.S. Cl. CPC A61K 31/357; A61K 31/365; A61K 31/4706; A61K 31/4741; Y02A 50/30

(57) ABSTRACT

The present invention describes a method of treating individuals suffering from microbial infections, including a parasitic disease such as malaria, by using an improved Artemisinin Combination Therapy (ACT), known as ActRx Tri-ACT Plus. The improved ACT therapy includes administering a combination of four drugs. In one embodiment of the present invention, the method includes administering to an individual a first composition comprising a therapeutically effective amount of an artemether spray sublingually. The individual is then administered a second composition, a therapeutically effective amount of artesunate. A third composition, an effective amount of berberine, or its pharmaceutically acceptable derivatives or salts is administered to the individual. A fourth composition, an effective amount of primaquine, or its pharmaceutically acceptable derivatives or salts is administered to the individual.

### REFERENCE TO RELATED APPLICATIONS

In accordance with 37 C.F.R. 1.76, a claim of priority is included in an Application Data Sheet filed concurrently herewith. Accordingly, the present invention is a continuation application of U.S. Pat. No. 14/201,740, filed on Mar. 7, 2014, which claims priority to U.S. Provisional Patent Application No. 61/774,664, entitled “ENHANCED ARTEMISININ-BASED COMBINATION THERAPY FOR TREATING PARASITIC MEDIATED DISEASE”, filed Mar. 8, 2013 and of U.S. Provisional Patent Application No. 61/878,848, entitled “ARTEMETHER SPRAY”, filed Sep. 17, 2013, and as a Continuation-in-Part of U.S. patent application Ser. No. 13/542,719, entitled “ARTEMISININ-BASED COMBINATION THERAPY FOR TREATING PARASITIC MEDIATED DISEASE”, filed Jul. 6, 2012.

### FIELD OF THE INVENTION

The present invention relates to microbial therapies, and particularly to a method of treating individuals suffering from a parasitic related disease using an improved Artemisinin based therapy, to a method of treating an individual suffering from malaria using an improved Artemisinin Combination Therapy (ACT); and more particularly to an enhanced method of treating an individual suffering from all forms of malaria from multiple parasite species using an improved Artemisinin Combination Therapy (ACT) adapted to prevent reoccurrence and minimize transmission stemming from untreated gametocytes in the liver.

### BACKGROUND OF THE INVENTION

Malaria is a serious and complex tropical parasitic disease spread by several species of mosquito. It is caused by a parasite from the Plasmodium genus, particularly Plasmodium falciparum, Plasmodium vivax, Plasmodium ovale, Plasmodium malariae, and Plasmodium knowlesi. The symptoms of malaria include fever, headaches, and vomiting usually after 10-15 days post mosquito bite. Left untreated, it may quickly develop into life-threatening complications. The Plasmodium parasites undergo complex life cycles once inside their human host. The life cycle starts with the mosquito bite of an infected female Anopheles mosquito. As a result of the bite, sporozoites are injected into the bloodstream, eventually reaching the liver. Once in the liver, the sporozoites multiply and release merozoites into the bloodstream where they invade the erythrocytes. In some infections, i.e. those caused by P. vivax, many parasites remain dormant in the liver. Those parasites in the liver can be reactivated, ultimately causing relapse. In addition to those remaining in the liver, many parasites can live and remain dormant in the gastrointestinal tract.

Malaria is a world wide problem with an estimated 500+ million cases in 2010. It is estimated that over 1.5 million people die every year from malaria, with most patients being children under the age of 5. A number of drugs ranging from natural drugs such as artemisinin and quinine, to synthetic drugs such as chloroquine, mefloquine, primaquine, halofantrine, amodiaquine, proguanil, and maloprim, have been developed to treat malaria. Despite the development of anti-malarial drugs, the number of infections and deaths related to the infection continue to rise. One factor resulting in the large number of malaria-related mortalities is the fact that Plasmodium falciparum, the deadliest malaria parasite, has acquired resistance against many available anti-malarial drugs. In fact, the World Health Organization (WHO) now prohibits the use of single compositions to fight the disease.

Therefore, what is needed in the art is an easy to administer method of treating individuals using an improved ACT methodology to individuals suffering from parasitic mediated disease, such as malaria. The method of treating individuals suffering from a parasitic mediated disease such as malaria, must be capable of treating all forms of the disease (uncomplicated, complicated and cerebral), must account for the multiple parasites species causing the disease, and must prevent relapse or reoccurrence. When used for malaria, the treatment must be able to treat all forms of malaria without the need to identify which species specific disease type, must not have any side effects, and must clear gametocytes to prevent reoccurrence of the disease.

### SUMMARY OF THE INVENTION

The present invention describes a therapy for individuals suffering from a parasitic infection based on an enhanced Artemisinin Combination Therapy (ACT). In contrast to most ACT therapies combination which utilize a dual drug therapy, the present invention describes a method which uses a combination of four drugs. In one embodiment of the present invention, the method includes administering to an individual a first composition. The first composition comprises a therapeutically effective amount of an artemisinin derivate or its salt, such as an artemether spray delivered sublingually. The individual is then administered a second composition. The second composition comprises of a therapeutically effective amount of a second artemisinin derivate or its salt. The second artemisinin derivate differs from the first composition and is preferably artesunate. A third anti-microbial composition is then administered to the individual. The third composition comprises of an effective amount of berberine, or its pharmaceutically acceptable derivatives or salts. The second and third compositions are administered to the individual for additional periods, such as for two or three days. A fourth anti-malarial composition is then administered to the individual. The fourth composition should be a compound that eliminates tissue infection (exo-erythrocytic, preferably in the liver) and therefore, prevents the development of blood (erythrocytic) forms of the parasite which are responsible for relapses in malaria. Preferably, the fourth composition comprises of an effective amount of Primaquine, or its pharmaceutically acceptable derivatives or salts. The fourth composition may be given to individuals on Day 4 as a one time dosage administration, or may be administered for a pre-determined period, up to for example 14 days, and preferably for 14 days. The novel combination therapy in accordance with the present invention may be referred to generally as TriAct Plus therapy.

In an alternative embodiment, the treatment is administered to an individual suffering from a disease transmitted by an arthropod, such as a mosquito or tick.

In an alternative embodiment, the treatment is administered to an individual suffering from a disease mediated by a eukaryotic protist of the genus Plasmodium.

In an alternative embodiment, the treatment is administered to an individual suffering from a disease mediated by Plasmodium falciparum, Plasmodium vivax, Plasmodium ovale, Plasmodium malariae, and Plasmodium knowlesi.

In an alternative embodiment, the treatment is administered to an individual suffering from a disease mediated by a combination of one or more of Plasmodium falciparum, Plasmodium vivax, Plasmodium ovale, Plasmodium malariae, and Plasmodium knowlesi.

In an alternative embodiment, the treatment is administered to an individual suffering from malaria.

In an alternative embodiment, the treatment is administered to an individual suffering from cerebral malaria.

In an alternative embodiment, the treatment is administered to an individual in order to prevent relapse or reoccurrence of a disease mediated by one or more of Plasmodium falciparum, Plasmodium vivax, Plasmodium ovale, Plasmodium malariae, and Plasmodium knowlesi.

When used for individuals suffering from, for example, Malaria, the TriAct Plus therapy can 1) be used as a universal treatment to cure for all four (4) Plasmodia species (Plasmodium falciparum, Plasmodium vivax, Plasmodium ovale, and Plasmodium malariae), 2) facilitates early administration of the treatment not requiring specie identification, thus preventing eventual complications of delayed treatment, 3) uses a delivery method for artemether via sublingual (spray) to treat complicated and severe forms of Malaria especially in the absence of parenteral forms and may serve as a treatment option regardless of the level of consciousness of the patient (cerebral malaria), even when treatment is critical in the most remote areas. This method eliminates the first pass effect (the drug bypasses the liver, thereby reaching the systemic circulation immediately) without sacrificing the serum concentration. The TriAct Plus therapy was shown to be effective in the reduction of fever and headaches, elimination all forms of parasites within two (2) days, and promotion of needle-free treatment. The TriAct Plus therapy demonstrated no incidence of recurrence, recrudescence, or relapse. The subjects receiving the study drugs did not experience any adverse reactions like hypoglycemia, palpitations and tinnitus which are typically observed with quinine use or its derivatives.

The present invention further describes an improved artemether based supplement and/or pharmaceutical composition which is administered as a sublingual spray. The supplement and/or pharmaceutical composition contains an Artemisinin derivative, an allergenic minimizing carrier, and a mucosal absorption enhancer. The present invention further relates to a method of manufacturing the artemether based supplement and/or pharmaceutical composition and the uses thereof for delivery to an individual in need thereof.

As used herein, the term “pharmaceutically acceptable excipient” or “pharmaceutically acceptable carrier,” generally refers to organic or inorganic materials, non-toxic, inert solid, semi-solid or liquid filler, diluent, encapsulating material or formulation auxiliary of any type which cannot react with active ingredients. The excipients include but are not limited to sugars, such as lactose, glucose and sucrose; starches such as corn starch and potato starch; cellulose and its derivatives such as sodium carboxymethylcellulose, ethylcellulose, and cellulose acetate; powered tragacanth; malt; gelatin; talc; stearic acids; magnesium stearate; calcium sulfate; cocoa butter and suppository waxes; vegetable oils, such as peanut oil, cotton seed oil, sesame oil, olive oil, corn oil and oil of theobroma; esters such as but not limited to, ethyl oleate and ethyl laurate; polyols such as propylene glycol, glycerine, sorbitol, mannitol and polyethylene glycol; agar; alginic acids; buffering agents such as but not limited to, magnesium hydroxide and aluminum hydroxide; pyrogen-free water; isotonic saline; and phosphate buffer solution; skim milk powder; as well as other non-toxic compatible substances used in pharmaceutical formulations. Wetting agents and lubricants such as sodium lauryl sulfate and magnesium stearate, as well as coloring agents, flavoring agents, sweetening agents, lubricants, releasing agents, perfuming agents, carriers, tabletting agents, stabilizers, antioxidants and preservatives can also be present.

As use herein, “Pharmaceutically-acceptable salt” refers to salts which retain the biological effectiveness and properties of compounds which are not biologically or otherwise undesirable. Pharmaceutically-acceptable salts refer to pharmaceutically-acceptable salts of the compounds, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

As used herein, the term “therapeutically effective amount” generally refers to an amount of an agent, for example the amount of a compound as an active ingredient, that is sufficient to effect treatment as defined herein when administered to an individual, such as a mammal, preferably a human in need of such treatment. A therapeutically effective amount of a compound, salt, analog, or derivative of the present invention will depend on a number of factors including, for example, the age and weight of the subject, the precise condition requiring treatment and its severity, the nature of the formulation, and the route of administration, and will ultimately be at the discretion of the attendant physician.

As used herein, the term “treat”, “treating” or “treatment” refers to the administration of therapy to a subject, particularly a mammal, more particularly a human, who already manifests at least one symptom of a disease to obtain a desired pharmacological and physiological effect. Such a subject includes an individual who is diagnosed as having a disease. The term may also include preventing the disease, i.e., causing the clinical symptoms of the disease not to develop in a mammal that may be exposed to or predisposed to the disease but does not yet experience or display symptoms of the disease, inhibiting the disease, i.e., arresting or reducing the development of the disease or its clinical symptoms, or relieving the disease, i.e., causing regression of the disease or its clinical symptoms.

Accordingly, it is an objective of the present invention to provide an enhanced Artemisinin Combination Therapy to individuals in need thereof.

It is a further objective of the present invention to provide an enhanced Artemisinin-based Combination Therapy to individuals suffering from a parasitic disease.

It is a further objective of the present invention to provide an enhanced Artemisinin-based Combination Therapy to individuals suffering from a parasitic disease transmitted by arthropods, such as a mosquito.

It is a still further objective of the present invention to provide an enhanced Artemisinin-based Combination Therapy to individuals suffering from a disease mediated by a eukaryotic protist of the genus Plasmodium.

It is a further objective of the present invention to provide an enhanced Artemisinin-based Combination Therapy incorporating a sublingual delivery step to individuals suffering from a parasitic disease.

It is yet another objective of the present invention to provide an enhanced Artemisinin-based Combination Therapy incorporating a sublingual delivery step to individuals suffering from a disease mediated by a eukaryotic protist of the genus Plasmodium.

It is a still further objective of the present to provide an enhanced Artemisinin-based Combination Therapy incorporating delivery of an artemisinin derivate using a sublingual, spray-based delivery route of administration to individuals suffering from malaria.

It is a further objective of the present invention to provide an enhanced Artemisinin-based Combination Therapy incorporating delivery of an artemisinin derivative using a sublingual, spray-based delivery route of administration in combination with oral delivery of a second, independent artemisinin derivative, a berberine, or its derivatives, and a medicament for killing parasites in body tissue, for individuals suffering from a parasitic disease.

It is a further objective of the present invention to provide an enhanced Artemisinin-based Combination Therapy incorporating delivery of an artemisinin derivative using a sublingual, spray-based delivery route of administration in combination with oral delivery of a second, independent artemisinin derivative, a berberine, or its derivatives, and a medicament for killing parasites in the liver, for individuals suffering from a parasitic disease.

It is a further objective of the present invention to provide an enhanced Artemisinin-based Combination Therapy incorporating delivery of an artemisinin derivative using a sublingual, spray-based delivery route of administration in combination with oral delivery of a second, independent artemisinin derivative, a berberine, or its derivatives, and a anti-malarial drug for killing malaria parasites in body tissue of individuals suffering from malaria.

It is yet another objective of the present invention to provide an improved Artemisinin-based Combination Therapy incorporating delivery of an artemisinin derivative using a sublingual, spray-based delivery route of administration in combination with oral delivery of a second, independent artemisinin derivative, a berberine, or its derivatives, and primaquine, or its salts for individuals suffering from a parasitic disease.

It is a further objective of the present invention to provide an enhanced Artemisinin-based Combination Therapy incorporating delivery of an artemisinin derivative using a sublingual, spray-based delivery route of administration in combination with oral delivery of a second, independent artemisinin derivative, a berberine, or its derivatives, and an anti-malarial for killing malaria parasites in liver of individuals suffering from malaria.

It is a further objective of the present invention to provide an enhanced Artemisinin-based Combination Therapy incorporating delivery of an artemisinin derivative using a sublingual, spray-based delivery route of administration in combination with oral delivery of a second, independent artemisinin derivative, a berberine, or its derivatives, and a anti-malarial drug for preventing relapses by acting against the liver stages of one or more malaria causing parasites of individuals.

It is a further objective of the present invention to provide an enhanced Artemisinin-based Combination Therapy incorporating delivery of an artemisinin derivative using a sublingual, spray-based delivery route of administration in combination with oral delivery of a second, independent artemisinin derivative, a berberine, or its derivatives, and primaquine, or its salts, for individuals suffering from malaria.

It is yet another objective of the present invention to provide an improved Artemisinin-based Combination Therapy incorporating delivery of an artemisinin derivative using a sublingual, spray-based delivery route of administration in combination with oral delivery of a second, independent artemisinin derivative, a berberine, or its derivatives, and primaquine, or its salts, for individuals suffering from cerebral malaria.

It is an objective of the present invention to provide an improved artemether based supplement and/or pharmaceutical composition for treating individuals suffering from disease.

It is a further objective of the present invention to provide an improved artemether based supplement and/or pharmaceutical composition for treating individuals infected with at least one parasite known to cause malaria.

It is yet another objective of the present invention to provide a spray artemether supplement and/or pharmaceutical composition for treating individuals suffering from disease.

It is a still further objective of the invention to provide an artemether supplement and/or pharmaceutical composition which can be delivered as a sublingual spray for treating individuals suffering from disease.

It is a further objective of the present invention to provide an artemether supplement and/or pharmaceutical composition which can be delivered as a sublingual spray for treating individuals suffering from disease and contains a carrier that minimizes the risk of an allergic response.

It is yet another objective of the present invention to provide an artemether supplement and/or pharmaceutical composition which can be delivered as a sublingual for treating individuals suffering from disease and contains a carrier that enhances mucosal absorption.

It is a still further objective of the invention to provide an artemether supplement and/or pharmaceutical composition which can be delivered as a sublingual spray for treating individuals suffering from disease and contains a carrier that minimizes the risk of an allergic response and enhances mucosal absorption.

Other objectives and advantages of this invention will become apparent from the following description taken in conjunction with any accompanying drawings wherein are set forth, by way of illustration and example, certain embodiments of this invention. Any drawings contained herein constitute a part of this specification and include exemplary embodiments of the present invention and illustrate various objects and features thereof.

### BRIEF DESCRIPTION OF THE FIGURES

FIG. 1 is a graph comparing individuals diagnosed with malaria and treated with the TriAct Plus therapy versus individuals diagnosed with malaria and treated using standard care protocol with respect to the number of days of positive malarial smear;

FIG. 2 is a graph comparing individuals diagnosed with malaria and treated with the TriAct Plus therapy versus individuals diagnosed with malaria and treated using standard care protocol with respect to the number of days of abnormal platelet count;

FIG. 3 is a graph comparing individuals diagnosed with malaria and treated with the TriAct Plus therapy versus individuals diagnosed with malaria and treated using standard care protocol with respect to the number of days of abnormal hemoglobin levels;

FIG. 4 is a graph comparing individuals diagnosed with malaria and treated with the TriAct Plus therapy versus individuals diagnosed with malaria and treated using standard care protocol with respect to the number of days of abnormal WBC count;

FIG. 5 is a graph comparing individuals diagnosed with malaria and treated with the TriAct Plus therapy versus individuals diagnosed with malaria and treated using standard care protocol with respect to the number of days of tacypnea;

FIG. 6 is a graph comparing individuals diagnosed with malaria and treated with the TriAct Plus therapy versus individuals diagnosed with malaria and treated using standard care protocol with respect to the number of days of hypotension;

FIG. 7 is a graph comparing individuals diagnosed with malaria and treated with the TriAct Plus therapy versus individuals diagnosed with malaria and treated using standard care protocol with respect to the number of days of altered sensorium;

FIG. 8 is a graph comparing individuals diagnosed with malaria and treated with the TriAct Plus therapy versus individuals diagnosed with malaria and treated using standard care protocol with respect to the number of days of abdominal pain;

FIG. 9 is a graph comparing individuals diagnosed with malaria and treated with the TriAct Plus therapy versus individuals diagnosed with malaria and treated using standard care protocol with respect to the number of days of vomiting;

FIG. 10 is a graph comparing individuals diagnosed with malaria and treated with the TriAct Plus therapy versus individuals diagnosed with malaria and treated using standard care protocol with respect to the number of days of nausea;

FIG. 11 is a graph comparing individuals diagnosed with malaria and treated with the TriAct Plus therapy versus individuals diagnosed with malaria and treated using standard care protocol with respect to the number of days of myalgia;

FIG. 12 is a graph comparing individuals diagnosed with malaria and treated with the TriAct Plus therapy versus individuals diagnosed with malaria and treated using standard care protocol with respect to the number of days of anorexia;

FIG. 13 is a graph comparing individuals diagnosed with malaria and treated with the TriAct Plus therapy versus individuals diagnosed with malaria and treated using standard care protocol with respect to the number of days of fever;

FIG. 14 is a graph comparing individuals diagnosed with malaria and treated with the TriAct Plus therapy versus individuals diagnosed with malaria and treated using standard care protocol with respect to the number of days with a headache;

FIG. 15 is a graph comparing individuals diagnosed with malaria and treated with the TriAct Plus therapy versus individuals diagnosed with malaria and treated using standard care protocol with respect to the number of days with reported chills.

### DETAILED DESCRIPTION OF THE INVENTION

While the present invention is susceptible of embodiment in various forms, there is demonstrated and will hereinafter be described a presently preferred, albeit not limiting, embodiment with the understanding that the present disclosure is to be considered an exemplification of the present invention and is not intended to limit the invention to the specific embodiments illustrated.

The present invention describes a novel combination therapy for the treatment of microbial infections, such as parasitic infections. As an illustrative example, the present invention may be useful in treating diseases mediated by arthropods and/or a Plasmodium based parasites causing malaria. The combination therapy in accordance with the present invention is based on Artemisinin Combination Therapy (ACT). In accordance with the World Health Organization (WHO) regulations for the use of artemisinin based treatments, such compounds are generally combined with other drugs to minimize resistance. The present invention uses a quad-therapy modality, using a unique combination of four compositions: artemether, artesunate, berberine, and primaquine. While most drugs currently used kill parasites in the blood stream, parasites in the gut and other tissues such as the liver remain where they or their eggs lodge and lay dormant for years. Such dormancy accounts for re-occurrence of the disease years later. Use of the quad-therapy may allow for killing of the parasites in the blood (artemesinin derivatives), the digestive tract (berberine), and the liver (primaquine). The quad-therapy in accordance with the present invention allows for quick administration to patients suffering cerebral malaria. The novel combination therapy may be referred to generally as TriAct Plus therapy, for the triple components plus primaquine that make up combination therapy.

Using this therapy, there is no need to make a determination of which type of malaria (cerebral, complicated, non-complicated, non-cerebral) or species specific the individual has, thereby minimizing the delay in treatment. While there are several diagnostic laboratory examinations used in the diagnosis of malaria, such as microscopy (thin and thick smears) or the Blood Smear for Malaria Parasite (BSMP), fluorescent microscopy, detection of parasite antigen, polymerase chain reaction, flow cytometry and rapid diagnostic test kits, these may not always be available in certain portions of the world and/or may result in increased delay in beginning treatment until the tests are performed and analyzed. The present invention minimizes the need for performing such tests. In addition, the quad-therapy in accordance with the present invention may allow for treating individuals suffering from malaria caused by one or more of Plasmodium falciparum, Plasmodium vivax, Plasmodium ovale, and Plasmodium malariae, and possibly Plasmodium knowlesi. Administering the first composition by a sublingual spray not only allows the drug to enter the patient's body rapidly, but allows delivery to patients who may not be able to receive dosages via pills and eliminates any problems associated with using needles (i.e. safety, disposal). Follow up doses of the second and third compositions ensure that the parasites are killed both in the blood and the digestive tract, thereby eliminating re-occurrence. Doses of the fourth composition ensure that the parasites are killed in the liver, thereby eliminating re-occurrence.

### Artemisinin and Artemisinin Derivatives

Artemisinin, having an IUPAC name of 3R,5aS,6R,8aS,9R,12S,12aR)-octahydro-3,6,9-trimethyl-3,12-epoxy-12H-pyrano[4,3-j]-1,2-benzodioxepin-10(3H)-one and also known as Qinhaosu, is a natural product derived from the Chinese herb Artemisia annua known to have anti-viral activities, see Efferth et al. The Antiviral Activities of Artemisinin and Artesunate. Clin. Inf. Dis. 2008: 47:804-11. It has a chemical structure of:

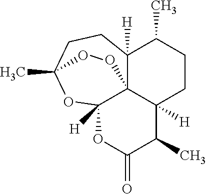 [SMILES: C[C@@H]1CC[C@H]2[C@@H](C)C(=O)O[C@@H]3O[C@]4(C)CC[C@@H]1[C@]32OO4]

Artemisinin is a sesquiterpene lactone containing an unusual peroxide bridge. This peroxide is believed to be responsible for the drug's mechanism of action. Few other natural compounds with such a peroxide bridge are known, see Artemisinin and a new generation of antimalarial drugs”. Education in Chemistry. July 2006 (see webpage, rsc.org/Education/EiC/issues/2006July/Artemisinin.asp). It is widely used for the treatment of malaria, particularly in combination with other drugs such as mefloquine (ASMQ), lumefantrine (such as sold under the trade name Coartem), amodiaquine (such as sold under the trade names Camoquin, Flavoquine, ASAQ), piperaquine (such as sold under the trade name Duo-Cotecxin), artemisinin and pyronaridine (such as sold under the trade name Pyramax). Artemisinin derivatives are typically used as prodrugs of the biologically active metabolite dihydroartemisinin, which is active during the stage when the parasite is located inside red blood cells. The mechanism through which artemisinin derivatives kill the parasite is believed to act via perturbing redox homeostasis in malaria parasites. Accordingly, such compositions may include free-radical production in the parasite food vacuole and inhibition of a parasite calcium ATPase. A key advantage of artemisinins is rapid action against all of the erythrocytic stages of the parasite, including transmissible gametocytes, resulting in a rapid clinical benefit and decreased transmission of malaria, see Rosenthal, Artesunate for Treatment of Severe falciparum Malaria, N. Engl J Med 2008, 358:1829-1836.

One of the disadvantages of artemisinin is its poor physical properties resulting in poor bioavailability, limiting its effectiveness. For example, the compound is sparingly soluble in either water or oils and thus not readily absorbable by the gastrointestinal tract. As a result, numerous semi-synthetic derivatives have been developed, including artesunate (water-soluble: for oral, rectal, intramuscular, or intravenous use), artemether (lipid-soluble: for oral, rectal or intramuscular use) dihydroartemisinin, artelinic acid, artenimol and artemotil. Accordingly, the Artemisinin derivatives include but are not limited to artesunate, dihydroartemisinin, dihydroartemisinin hemisuccinate, dihydrodroartemisinin succinate, sodium artesunate, stabilized forms of artesunate, stabilized forms of sodium artesunate, dihydroartemisitene dimers as described in U.S. Pat. No. 7,098,242, amino-functionalized 1,2,4-trioxanes as described in U.S. Pat. No. 7,071,226, artemisinin endoperoxides as described in U.S. Pat. No. 6,984,640, spiro and dispiro 1,2,4-trioxolane anti-malarials as described in U.S. Pat. No. 6,906,205, mixed steroidal 1,2,4,5-tetraoxane compounds as described in U.S. Pat. No. 6,906,098, arteether as described in U.S. Pat. No. 6,750,356, substituted 1,2,4-trioxanes as described in U.S. Pat. No. 6,737,438, Artemisia annua extracts as described in U.S. Pat. No. 6,685,972, artemether as described in U.S. Pat. No. 6,683,193, trioxane derivatives based on artemisinin as described in U.S. Pat. No. 6,649,647, trioxane dimer compounds as described in U.S. RE38,117, conjugates of artelinic acid as described in U.S. Pat. No. 6,461,603, arteethers from dihydroartemisinin as described in U.S. Pat. No. 6,346,631, artemisinine or artemisinene derivatives as described in U.S. Pat. No. 6,306,896, C-10 carbon substituted artemisinin-like trioxane compounds as described in U.S. Pat. No. 6,160,004, water-soluble trioxanes as described in U.S. Pat. No. 6,136,847, alpha arteether as described in U.S. Pat. No. 6,127,405, artemisinin dimers as described in U.S. Pat. No. 5,856,351, (+)-deoxoarteminisinin and analogs of (+)-deoxoartemisinin as described in U.S. Pat. No. 5,225,562, and 10-substituted ether derivatives of dihydroartemisinin as described in U.S. Pat. No. 5,225,427, as well as its salts or other derivatives thereof as known to one of skill in the art.

Artemether is a methyl-ether derivative of dihydroartemisinin derived from artemisinin. It has an IUPAC chemical name of (+)-(3-alpha,5a-beta,6-beta,8a-beta,9-alpha,12-beta,12aR)-decahydro-10-methoxy-3,6,9-trimethyl-3,12-epoxy-12H-pyrano(4,3-j)-1,2-benzodioxepin, having a chemical formula:

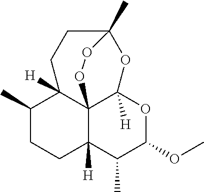

Artemether is known to be effective against blood schizots of several malaria causing parasites, and is used as an ACT drug.

Artesunate, also known as dihydroartemisinin hemisuccinate and its salts, has a IUPAUC chemical name of 3R,5aS,6R,8aS,9R,10S,12R,12aR)-Decahydro-3,6,9-trimethyl-3,12-epoxy-12H-pyrano(4,3-j)-1,2benzodioxepin-10-ol hydrogen succinate, with a chemical formula of:

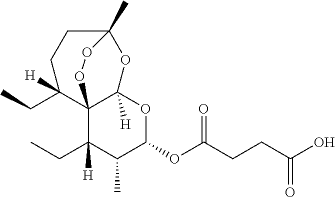

Artesunate is used primarily as treatment for malaria.

### Dosage for Artemisinin, Artemisinin Salts and Derivatives

The oral dosage range for artemisinin, artemisinin salts and derivatives is between about 1 mg to about 1,500 mg per dose administered one to three times daily. More preferably, the oral dosage range for Artemisinin, Artemisinin salts and derivatives is between about 20 mg to about 250 mg per dose if taken one to three times daily. Most preferably, the oral dosage ranges for artemisinin, artemisinin salts and derivatives is between about 40 mg to about 100 mg per dose taken one to three times daily.

### Berberine, Salts Thereof, and Berberine Derivatives

Berberine is a quaternary ammonium salt from the protoberberine group of isoquinoline alkaloids. It has a IUPAC name of (5,6-Dihydro-9,10-dimethoxybenzo[g]-1,3-benzodioxolo[5,6-a]quinolizinium), with a chemical formula of:

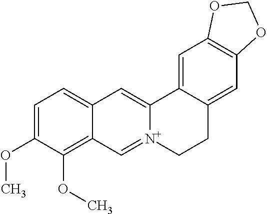 [SMILES: COc1ccc2cc3[n+](cc2c1OC)CCc1cc2c(cc1-3)OCO2]

It is found in various plant species of Berberis (e.g. Berberis aquifolium (Oregon grape), Berberis vulgaris (Barberry), and Berberis aristata (Tree Turmeric)), as well as other plant families, including but not limited to Hydrastis canadensis (Goldenseal), Phellodendron amurense (Amur Cork Tree, Huang Bai, Huang Po, Po Mu) and Coptis chinensis (Chinese Goldthread, Huang-Lian, Huang-Lien), and Tinospora cordifolia, and to a smaller extent in Argemone mexicana (Prickly Poppy) and Eschscholzia californica (Californian Poppy), Rhizoma coptidis Huanglian Jiedu decoction, San-Huang-Xie-Xin-Tang, Xietianwu, Gegen Quinlian, and Shizhu. Berberine is primarily isolated from the roots, rhizomes, stems, and bark. Berberine has been used for medical use in both Ayurvedic and Chinese medicines, and recently has been tested for antibacterial activity and use in diarrhea, prostate cancer and diabetes. It is generally considered to be non-toxic and shows no genotoxic activity.

In accordance with the present invention, berberine, berberine derivatives, or its salts may include, but not limited to, berberine alkaloid, berberine base, berberine hydrochloride, berberine, berberrubine, coreximine, tetrahydropalmatine, jatrorrhizine, 13-hydroxyberberine chloride, coralyne, coralyne chloride, 7,8-dihydro-13-methylberberine, berberine acetone, 13-allylberberine, palmatine, 13-benzylberberine, tetrahydroberberine,tetrahydroprotoberberine 8-cyanodihydroberberine, dimeric protoberberine alkaloids, demethylated protoberberine alkaloids, quaternary protoberberine alkaloids, protoberberine and protoberberine alkaloids, the salts of berberine, including berberine hydrochloride, berberine chloride, berberine sulfate, berberine tannate and other salts known to one of skill in the art.

### Dosage Range for Berberine, its Salts and Derivatives

The oral dosage range of berberine is from about 50 mg to about 1,500 mg administered in a single dose two to three times daily, not to exceed 4,500 mg per day. More preferably, the dose of berberine is about 100 mg to about 1,000 mg in a single human dose not to exceed 3,000 mg per day, taken one to three times daily. The most preferable dosage range for a single dose of berberine is about 200 to about 500 mg taken one to three times daily.

### Primaquine, and Salts Thereof

Primaquine is an 8-aminoquinoline anti-protozoal agent which is highly active against exo-erythrocytic stages of Plasmodium vivax, Plasmodium ovale and against the primary exo-erythrocytic stages of Plasmodium falciparum. Primaquine is also highly active against gametocytes of Plasmodia, especially Plasmodium falciparum. It has the chemical formula of:

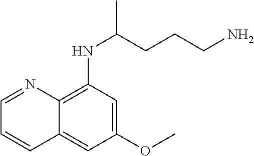 [SMILES: COc1cc(NC(C)CCCN)c2ncccc2c1]

Primaquine is readily absorbed from the gastro-intestinal tract and extensively distributed into body tissues. Peak plasma concentration occurs about 1 to 3 hours after a dose is taken and then rapidly diminishes with a reported elimination half life of 3 to 6 hours. Primaquine is rapidly metabolized in the liver, its principal metabolite being carboxyprimaquine. In accordance with the present invention, the preferred form of the composition is its salt derivative, preferably the phosphate salt, primaquine phosphate. Primaquine phosphate may be given at orally as a tablet in dosages of 0.5-0.75 mg-base per kilogram if based on weight. Primaquine phosphate may be given at daily dosages of 15-45 mg as a single dosage administration and 7.5 to 15 mg/day for 14 days if based on age. Dosages and administration of primaquine phosphate can vary depending on the desired treatment needed as a result of the type of Plasmodium species.

TABLE 1
Delivery Schedule for Uncomplicated Malaria (P. falciparum/
P. malariae): Primaquine phosphate 15 mg/tablet.

| | | | | |
|---|---|---|---|---|
| Weight- | use 0.75 mg- | | | |
| based | base per | | | |
| | kilogram | | | |
| | single dose | | | |
| Age-based | ≥12 year old | 7-11 year old | 4-6 year old | 1-3 year old. |
| | 3 Primaquine | 2 Primaquine | 1 Primaquine | ½ |
| | phosphate a | phosphate | phosphate | Primaquine |
| | tablets | tablets | tablet | phosphate |
| | single dose | single dose | single dose | single dose |

After the administration of the artemether, artesunate, and berberine on Days 1 to 3, on Day 4 individuals may be given Primaquine phosphate as a single dose.

TABLE 2
Uncomplicated Malaria (P. vivax/P. ovale): Primaquine
phosphate 15 mg/tablet administered at Day 4 to Day 17.

| | | | | |
|---|---|---|---|---|
| Weight-based | use 0.5 mg- | | | |
| | base per | | | |
| | kilogram | | | |
| | body | | | |
| | weight/day × | | | |
| | 14 days | | | |
| Age-based | ≥12 year old | 7-11 year old | 4-6 year old | 1-3 year old. |
| | 1 tablet of | ¾ tablet of | ½ tablet of | ½ tablet of |
| | Primaquine | Primaquine | Primaquine | Primaquine |
| | phosphate | phosphate | phosphate | phosphate |
| | Daily | Daily | Daily | Daily |

After the administration of the artemether, artesunate, and berberine on Days 1 to 3, on Days 4 to 17 individuals may be given Primaquine phosphate.

TABLE 3
Mixed Infection (P. falciparum + P. vivax) or
(P. vivax + P. malariae) or (P. falciparum +
P. vivax + P. malariae): Primaquine phosphate
15 mg/tablet administered at Day 4 to day 17.

| | | | | |
|---|---|---|---|---|
| Weight-based | use 0.5 mg- | | | |
| | base per | | | |
| | kilogram | | | |
| | body | | | |
| | weight/day × | | | |
| | 14 days | | | |
| Age-based | ≥12 year old | 7-11 year old | 4-6 year old | 1-3 year old. |
| | 1 tablet of | ¾ tablet of | ½ tablet of | ½ tablet of |
| | Primaquine | Primaquine | Primaquine | Primaquine |
| | phosphate | phosphate | phosphate | phosphate |
| | Daily | Daily | Daily | Daily |

After the administration of the artemether, artesunate, and berberine on Days 1 to 3, on Days 4 to 17 individuals may be given Primaquine phosphate.

TABLE 4
Mixed Infection (P. falciparum +
P. malariae). Primaquine phosphate 15 mg/tablet.

| | | | | |
|---|---|---|---|---|
| Weight- | use 0.75 mg- | | | |
| based | base per | | | |
| | kilogram | | | |
| | single dose | | | |
| Age-based | ≥12 year old | 7-11 year old | 4-6 year old | 1-3 year old. |
| | 3 Primaquine | 2 Primaquine | 1 Primaquine | ½ |
| | phosphate | phosphate | phosphate | Primaquine |
| | tablets | tablets | tablet | phosphate |
| | single dose | single dose | single dose | single dose |

After the administration of the artemether, artesunate, and berberine on Days 1 to 3, on Day 4 individuals may be given Primaquine phosphate as a single dose.

The present invention is further described by the following non-limiting examples. The following examples illustrate embodiments, albeit preferred embodiments, of routes of administration and forms of the compositions. While the preferred forms and/or routes are described, other forms, such as but not limited to tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions formulations for intravesical administration. The compositions may also be developed for inhalational, oral, rectal, vaginal, parenteral, topical, transdermal, pulmonary, intranasal, buccal, ophthalmic, intrathecal, intravenous or any other route of administration

The dosage form of the present invention may include either immediate or controlled release forms. Each composition can be formulated and manufactured by procedures known to one of skill in the art, and may include 100% composition or a mixture of one or more pharmaceutically acceptable excipients or pharmaceutically acceptable carriers.

For use of sublingual sprays, liquid artemether can be prepared as a spray form using, for example, microfluidization process as described in U.S. Pat. No. 6,861,066. The artemether spray can be prepared as a sublingual nano-spray.

The present invention further describes an alternative sublingual spray, artemether based supplement and/or pharmaceutical composition, a method of manufacturing the improved artemether based supplement and/or pharmaceutical composition, and the use thereof for delivery to an individual in need thereof. The supplement and/or pharmaceutical composition contains an active compound such as an Artemisinin derivative, a carrier which reduces the risk of an allergic reaction when administered to an individual, and a mucosal absorption enhancer. In a preferred embodiment, the sublingual spray supplement and/or pharmaceutical composition includes artemether, a pharmaceutically non-allergenic acceptable carrier, such as neutral oil, including but not limited to olive oil (obtained from the fruit of Olea europaea), and a quaternary ammonium salt such as Benzalkonium Chloride which acts as a mucosal absorption enhancer.

The present invention includes an improved artemether supplement and/or pharmaceutical composition utilizing a carrier which reduces the risk of an allergic reaction when administered to an individual susceptible to food based allergies. While Artemisinin and Artemisinin derivatives for use in the treatment of malaria is known in the art, such compositions are prepared using Arachis (peanut) oil. The use of such oil presents a problem for any supplement or pharmacological routes of administration that require delivery within the oral cavity, such as the mouth whereby the active ingredient is required to diffuse into the circulatory system through tissues under the tongue. As a common trigger to those individuals suffering food induced allergic reactions, using peanut oil as a carrier for sublingual sprays increases the risk of severe injury and even fatalities.

To minimize the risk of potential allergic reactions, the supplement and/or pharmaceutical composition in accordance with the present invention uses olive oil as a carrier for the Artemether as there is no known cases of hypersensitivity to olive oil when taken by mouth.

### Mucosal Absorption Enhancer

Quaternary ammonium salts may be used to form the supplement and/or pharmaceutical composition. Such quaternary ammonium salts include those commonly used and considered as safe for human use. Such compounds are typically tetrasubstituted ammonium salts in which the substituent groups are preferably hydrocarbon compounds attached to the nitrogen by an N—C bond, and selected from the group consisting of substituted and unsubstituted, saturated and unsaturated, aliphatic and aromatic, and branched and normal chain groups. In all cases the nitrogen atom is pentavalent and is in the positively charged portion of the molecule, thus quaternary ammonium salts are cationic electrolytes.

Preferably, the mucosal absorption enhancer of this invention is benzalkonium chloride, also known as alkyl dimethylbenzyl ammonium chloride, alkyldimethyl (phenylmethyl), Quaternary Ammonium Chloride, Ammonyx, and Roccal. Benzalkonium chloride has a chemical formula of:

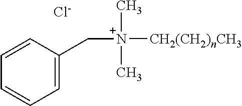

Benzalkonium chloride is commercially available in suitable form from a number of sources, including Sigma Aldrich Chemical Co. Benzalkonium chloride used in an amount effective to serve as a membrane enhance as well as a preservative. The Benzalkonium chloride may be used at a final concentration of between about 0.001% and about 0.1%, by weight, and preferably between about 0.005% and about 0.05%, based on the weight of the composition, and more preferably at 0.007% based on the weight of the composition.

Tables 5-9 provide various illustrative examples of the spray artemether supplement and/or pharmaceutical composition in accordance with the present invention.

TABLE 5
Sublingual Spray

| Component | Concentration |
|---|---|
| Artemether | Between about 5.0 mg and 80.0 |
| | mg by weight |
| Carrier | As needed |
| Membrane Enhancer | 0.001%-0.05% by weight |
| Preservative | 0.001%-0.05% by weight |

TABLE 6
Sublingual Spray

| Component | Concentration |
|---|---|
| Artemether | Between about 5.0 mg and 80.0 |
| | mg by weight |
| Non-allergenic Acceptable | As needed |
| Carrier | |
| Membrane Enhancer | 0.001%-0.05% by weight |
| Preservative | 0.001%-0.05% by weight |

TABLE 7
Sublingual Spray

| Component | Concentration |
|---|---|
| Artemether | Between about 5.0 mg and 80.0 |
| | mg by weight |
| Non-allergenic Acceptable | As needed |
| Carrier: Olive Oil | |
| Quaternary ammonium salts | 0.001%-0.05% by weight |

TABLE 8
Sublingual Spray

| Component | Concentration |
|---|---|
| Artemether | Between about 30.0 mg and |
| | about 80.0 mg by weight |
| Non-allergenic Acceptable | As needed |
| Carrier: Olive Oil | |
| Benzalkonium Chloride | 0.001%-0.05% by weight |

TABLE 9
Sublingual Spray

| Component | Concentration |
|---|---|
| Artemether | About 80.0 mg by weight |
| Non-allergenic Acceptable | As needed |
| Carrier: Olive Oil | |
| Benzalkonium Chloride | 0.007% by weight |

### Example of Method of Preparing Sublingual Artemether Spray, Single Dose 80 mg

To prepare the artemether sublingual spray, powdered artemether was obtained. In preparing single spray dosage, 700 mL of extra virgin olive oil was heated to 130 degree in a one liter beaker. The olive oil was cooled to 70 degrees. Once at 70 degrees, 80 grams of the artemether powder was added. The 80 grams of the artemether powder was slowly added to the cooled olive oil under continuous stirring until the powder was dissolved, forming a clear solution. To the artemether in solution, 0.7 ml of 10% Benzalkonium Chloride was added under continuous stirring. The artemether solution was allowed to cool to room temperature. Concentration of artemether was validated using HPLC.

Preferably, the artemether solution described above is packaged in a single dose delivery system. such as an atomizing spray pump as described in U.S. Pat. No. 6,126,038, where the patient can simply spray the composition to the desired place, i.e. under the tongue, until the bottle is completely discharged.

### Treatment Protocols

### EXAMPLE 1

### Method of Treating an Individual Suffering From a Disease Mediated by a Parasite

A method of treating an individual suffering from disease mediated by a parasite, preferably plasmodial parasites which cause malaria comprises the steps of (1) administering to a mammal, preferably a human, a first composition, the first composition comprising a therapeutically effective amount of an artemisinin derivative, preferably artemether at 50-80 mg per dose, preferably 60 mg (2) administering to the mammal a second composition, the second composition comprising a therapeutically effective amount of a second artemisinin derivate, the second artemisinin derivate differing from the first composition, and preferably being artesunate or its pharmaceutically acceptable derivatives at 1 mg-1500 mg per individual dose, preferably about 20 mg to about 250 mg per dose, administered three times daily, and most preferably about 40 mg to about 100 mg per dose administered three times daily; and (3) administering to a mammal a third composition, the third composition comprising a therapeutically effective amount of berberine, or its pharmaceutically acceptable derivatives at 1 mg to about 1500 mg per individual dose, particularly about 100 mg to about 1000 mg per dose three times a day, and more specifically about 200 mg to about 400 mg per dose, two to three times a day, and (4) administering to a mammal a fourth composition, the fourth composition comprising a therapeutically effective amount of primaquine, or its pharmaceutically acceptable derivatives at about 7 mg to about 45 mg per day, or for one day, or a period of up to 14 days, and preferably 14 days, if dosage determined by age, and about 0.5-0.75 mg per kg body weight per day or for one day, or a period of up to 14 days, and preferably 14 days, if dosage determined by body weight.

The following describes an illustrative, albeit preferred delivery treatment schedule:
Day 1: Delivery of artemether-single dose.
   Delivery of artesunate and berberine tablets, dosage according to user age and/or weight every 12 hours.
Day 2: Delivery of artesunate and berberine tablets, dosage according to user age and/or weight every 24 hours.
Day 3: Delivery of artesunate and berberine tablets, dosage according to user age every 24 hours.
Day 4: Delivery of artesunate and berberine tablets, dosage according to user age every 24 hours.
   Delivery of primaquine tablets, according to user age or weight, administration may be on Day 5 only or daily for a period of 14 days.
Day 5-17: If required, delivery of primaquine tablets, according to user age or weight,

### EXAMPLE 2

### Treatment for Adult Humans Over 165 Pounds (75 Kg) Having a Parasitic Infection Using Sublingual Delivery of an Artemether Solution: Malaria

A method of treating an individual suffering from malaria, including acute, chronic, or cerebral malaria, comprises the steps of (1) administering to an adult mammal, preferably a human, a first composition, the first composition comprising a therapeutically effective amount of artemether; (2) administering to the adult mammal a second composition, the second composition comprising a therapeutically effective amount of artesunate or its pharmaceutically acceptable derivatives; (3) administering to the adult mammal a third composition, the third composition comprising a therapeutically effective amount of berberine, or its pharmaceutically acceptable derivatives; and (4) administering to the adult mammal a fourth composition, the fourth composition comprising a therapeutically effective amount of primaquine, or its pharmaceutically acceptable derivatives or salts.

In a preferred embodiment, the method of treatment can be facilitated by the use of a kit comprising a sublingual spray containing artemether solution as a single dosage unit spray bottle, a first packet containing artesunate tablets, a second packet containing berberine tablets, and a third packet containing primaquine, or its pharmaceutically acceptable derivatives or salts. An adult human, over 165 pounds (75 Kg) suffering from malaria, including acute, chronic, or cerebral malaria, begins the method in accordance with the present invention by administering the first loading dose of the artemether. The artemether is delivered to the individual using a sublingual spray bottle containing 60 mg of artemether solution per bottle. Use of a spray offers advantages over injectable form by providing a method of delivery that is easy to administer, does not require trained medical professionals such as a doctor or nurse, and avoids the problems associated with needles, such as fear of spreading disease such as HIV or Hepatitis C and proper disposal. In addition, sublingual drug delivery, particularly sprays, allows the active ingredients improved absorption and enhanced bioavailability.

In use, the user simply removes the bottle from the transportation/delivery packaging and displaces any safety features, such as any tamper proofing outside wrapping, secured to the bottle. After removing the seal from the spray bottle, the adult individual lifts his/her tongue and pumps the spray bottle with artemether solution into the mouth so the solution is delivered to the area under the tongue. The bottle remains in that position until the contents of the bottle are fully delivered to the area under the tongue. The liquid is preferably held under the tongue for a predetermined time period, preferably 30 seconds. Any liquid remaining in the user's mouth is then swallowed. After waiting for a predetermined time period, such as 1 minute to 1 hour, and more preferably 30 minutes the user administers the second composition comprising a dosage of artesunate, such as 3×50 mg tablets for the first 12 hours, followed by a repeat dosage the next 12 hours (300 mg per day). The user then takes 2 tablets every 24 hours (100 mg) for 3 additional days. The user administers the third composition comprising a dosage of berberine, such as 2×400 mg tablets for the first 12 hours followed by a repeat dosage the next 12 hours (1600 mg per day). The user then takes 2 tablets every 24 hours (800 mg) for 3 additional days. Alternatively, the second and third compositions can be administered simultaneously with the first composition. The user also administers the fourth composition comprising a dosage of primaquine, or its pharmaceutically acceptable derivatives or salts, preferably primaquine phosphate. Preferably, administration of primaquine, or its pharmaceutically acceptable derivatives or salts is performed on Day 4 of the treatment and given as a single dose, according to the following, primaquine, administered to a patient by weight of 0.75 mg base per kilogram single dose as 1 one day administration. If the weight of the individual is unknown, primaquine tablets of 15 mg/tablet is administered based on an age based dosage. For individuals 12 years old or older, 3 primaquine tablets are given as a single dose administration. Depending on the need and/or type of Plasmodium species, multiple dosages may be given to the individual from Day 4 to Day 17, for a total of 14 days. For administration of primaquine for a period of 14 days, if the weight if the individual is known, primaquine is administered using 0.5 mg base per kilogram body weight for 14 days. If the weight of the individual is unknown, primaquine tablets, 15 mg/tablet, is administered based on an age based dosage. For individuals 12 years old or older, 1 primaquine tablet is administered daily for 14 days.

Preferably, the method of treating an individual suffering from malaria is performed by a procedure in which the first, second, third and fourth compositions are delivered to the infected individual without identifying the Plasmodium species affecting said individual. The method of treating an individual suffering from malaria includes the step of the combined drugs clearing all forms of the Plasmodium from the patient.

### EXAMPLE 3

### Treatment for Adult Human 66 (30 kg)-165 Pounds (75 Kg) Suffering a Parasitic Infection Using Sublingual Delivery of an Artemether Solution: Malaria

In a preferred embodiment, the method of treatment can be facilitated by the use of a kit comprising a sublingual spray containing artemether solution as a single dosage unit spray bottle, a first packet containing artesunate tablets, and a second packet containing berberine tablets. An adult human 66 (30 kg)-165 pounds (75 Kg) suffering from malaria, including acute, chronic, or cerebral malaria, begins the method in accordance with the present invention by administering the first loading dose of artemether. The artemether is delivered to the individual using a sublingual spray bottle containing 60 mg artemether solution per bottle. The user simply removes the bottle from the transportation/delivery packaging and displaces any safety features, such as outside wrapping to indicate tampering, secured to the bottle. After removing the seal from the spray bottle, the adult individual lifts his/her tongue and pumps the spray bottle with artemether solution into the mouth so the solution is delivered to the area under the tongue. The bottle remains in that position until the contents of the bottle are fully delivered to the area under the tongue. The liquid is preferably held under the tongue for a predetermined time period, preferably 30 seconds. Any liquid remaining in the user's mouth is then swallowed. After waiting for a predetermined time period, such as 1 minute to 1 hour, and more preferably 30 minutes, the user administers the second composition comprising a dosage of artesunate, such as 2×50 mg tablets for the first 12 hours, followed by a repeat dosage the next 12 hours (200 mg per day). The user then takes 1 tablet every 24 hours (50 mg) for 3 additional days. The user administers the third composition comprising a dosage of berberine, such as 1×400 mg tablets for the first 12 hours, followed by a repeat dosage the next 12 hours (800 mg per day). The user then takes 1 tablet every 24 hours (400 mg) for 3 additional days. Alternatively, the second and third compositions can be administered simultaneously with the first composition. The user also administers the fourth composition comprising a dosage of primaquine, or its derivatives or salts, preferably primaquine phosphate. Preferably, administration of primaquine is performed on Day 4 of the treatment and given as a single dose, according to the following, primaquine administered to a patient by weight of 0.75 mg base per kilogram single dose as 1 one day administration. If the weight of the individual is unknown, primaquine tablets of 15 mg/tablet is administered based on an age based dosage. For individuals 12 years old or older, 3 primaquine tablets are given as a single dose administration. Depending on the need and/or type of Plasmodium species, multiple dosages may be given to the individual from Day 4 to Day 17, for a total of 14 days. For administration of primaquine for a period of 14 days, if the weight if the individual is known, primaquine is administered using 0.5 mg base per kilogram body weight for 14 days. If the weight of the individual is unknown, primaquine tablets, 15 mg/tablet, is administered based on an age based dosage. For individuals 12 years old or older, 1 primaquine tablet is administered daily for 14 days.

Preferably, the method of treating an individual suffering from malaria is performed by a procedure in which the first, second, third and fourth compositions are delivered to the infected individual without identifying the Plasmodium species affecting said individual. The method of treating an individual suffering from malaria includes the step of the combined drugs clearing all forms of the Plasmodium from the patient.

### EXAMPLE 4

### Treatment for Human Child 33 Pounds (15 kg)-66 Pounds (30 Kg) Suffering a Parasitic Infection Using Sublingual Delivery of an Artemether Solution: Malaria

For children or individual in the range of between 33 pounds (15 kg)-66 pounds (30 Kg) suffering from malaria, including acute, chronic, or cerebral malaria, the treatment is the same as described in Example 3 for compositions 1-3. The user also administers the fourth composition comprising a dosage of primaquine, or its derivatives or salts, preferably primaquine phosphate. Preferably, administration of primaquine is performed on Day 4 of the treatment and given as a single dose, according to the following, primaquine administered to a patient by weight of 0.75 mg base per kilogram single dose as 1 one day administration. If the weight of the individual is unknown, primaquine tablets of 15 mg/tablet is administered based on an age based dosage. For individuals between 7 years old and 11 years old, 2 primaquine tablets are given as a single dose administration. For individuals between 4 years old and 6 years old, 1 primaquine tablets are given as a single dose administration. Depending on the need and/or type of Plasmodium species, multiple dosages may be given to the individual from Day 4 to Day 17, for a total of 14 days. For administration of primaquine for a period of 14 days, if the weight if the individual is known, primaquine is administered using 0.5 mg base per kilogram body weight for 14 days. If the weight of the individual is unknown, primaquine tablets, 15 mg/tablet, is administered based on an age based dosage. For individuals between 7 years old and 11 years old, ¾ of the primaquine tablet is administered daily for 14 days. For individuals between 4 years old and 6 years old, ½ of the primaquine tablet is administered daily for 14 days.

Preferably, the method of treating an individual suffering from malaria is performed by a procedure in which the first, second, third and fourth compositions are delivered to the infected individual without identifying the Plasmodium species affecting said individual. The method of treating an individual suffering from malaria includes the step of the combined drugs clearing all forms of the Plasmodium from the patient.

### EXAMPLE 5

### Treatment for Infants and Toddlers 33 Pounds Up to 3 Years Old (Under 15 kg) Suffering a Parasitic Infection Using Sublingual Delivery of an Artemether Solution: Malaria

For infants and toddlers 33 pounds up to 3 years old (under 15 kg) suffering from malaria, including acute, chronic, or cerebral malaria, the treatment is similar to Example 4. The dosage delivered to the user includes artemether delivered using a sublingual spray bottle containing 60 mg artemether solution per bottle, 25 mg artesunate per dosage, 1 dose every 12 hours for the first day (50 mg total per day) followed by 1 dose (25 mg) every day for three days, and 1×200 mg berberine dose every 12 hours for the first day (400 mg total per day) followed by 1 dose (200 mg) every day for three days. For infants and toddlers, the tablets can be ground up and mixed with liquids that allow delivery to the user, such as milk, juice, and syrups. The user also administers the fourth composition comprising a dosage of primaquine, or its derivatives or salts, preferably primaquine phosphate. Preferably, administration of primaquine is performed on Day 4 of the treatment and given as a single dose, according to the following, primaquine administered to a patient by weight of 0.75 mg base per kilogram single dose as 1 one day administration. If the weight of the individual is unknown, primaquine tablets of 15 mg/tablet is administered based on an age based dosage. For individuals between 1 years old and 3 years old, ½ of a primaquine tablet is given as a single dose administration. Depending on the need and/or type of Plasmodium species, multiple dosages may be given to the individual from Day 4 to Day 17, for a total of 14 days. For administration of primaquine for a period of 14 days, if the weight if the individual is known, primaquine is administered using 0.5 mg base per kilogram body weight for 14 days. If the weight of the individual is unknown, primaquine tablets, 15 mg/tablet, is administered based on an age based dosage. For individuals between 1 years old and 3 years old, ½ of the primaquine tablet is administered daily for 14 days.

Preferably, the method of treating an individual suffering from a malaria is performed by a procedure in which the first, second, third and fourth compositions are delivered to the infected individual without identifying the Plasmodium species affecting said individual. The method of treating an individual suffering from malaria includes the step of the combined drugs clearing all forms of the Plasmodium from the patient.

### EXAMPLE 6

### Treatment for Individuals Suffering a Parasitic Infection Using Injectable Delivery of Artemether Solution an Artemether Solution: Malaria

A method of treating an individual suffering from malaria comprises the steps of (1) administering to a mammal, preferably a human, a first composition, the first composition comprising a therapeutically effective amount of artemether; (2) administering to the mammal a second composition, the second composition comprising a therapeutically effective amount of a artesunate or its pharmaceutically acceptable derivatives; (3) administering to the mammal a third composition, the third composition comprising a therapeutically effective amount of berberine, or its pharmaceutically acceptable derivatives; and (4) and administering to the mammal a fourth composition, the fourth composition comprising a therapeutically effective amount of primaquine, or its pharmaceutically acceptable derivatives or salts, preferably primaquine phosphate.

Preferably, the method of treating an individual suffering from malaria is performed by a procedure in which the first, second, third and fourth compositions are delivered to the infected individual without identifying the Plasmodium species affecting said individual. The method of treating an individual suffering from malaria includes the step of the combined drugs clearing all forms of the Plasmodium from the patient.

Study using TriAct therapy on individuals diagnosed with malaria from all four types Plasmodium species.

Two hundred twenty (220) individuals with confirmed cases of Malaria by direct microscopy were enrolled and randomized into two groups, Group A, Species Specific Standard of Care using World Health Organization (WHO) guidelines, and Group B, TriAct Plus therapy. Patients in both groups were evaluated using clinical and laboratory parameters for comparison. The standard of care used was based on WHO Standards and the Philippines Department of Health Guidelines for treatment of malaria: inducing for uncomplicated P. flaciparium malaria, artemether-lumefantrine (AL) (20/120 mg) combination; for uncomplicated P. flaciparium, quinine sulfate in combination with either tetracycline or doxycycline or clindamtcin; for uncomplicated P. vivax/P. ovale, chloriquine, 150 mg; for uncomplicated P. malariae chloriquine, 150 mg; for mixed infection (P. falciparum and P. vivax), artemether-lumefantrine (AL) (20/120 mg) combination; for mixed infection (P. falciparum and P. malariae, artemether-lumefantrine (AL) (20/120 mg); mixed infection (P. malariae and P. vivax), chloriquine, 150 mg; for mixed infection (P. falciparum and P. malariae and P. vivax) artemether-lumefantrine (AL) (20/120 mg) combination; severe malaria, P. falciparum, quinine IN plus artemether-lumefantrine (AL) (20/120 mg).

### Materials and Methods

The study carried out was a prospective, randomized, controlled multi-center study enrolling two hundred twenty (220) subjects stratified into two treatment groups, Group A, WHO Standard Malaria Treatment and Group B, TriAct Plus therapy in several health facilities in the Philippines. Subjects included individuals of ages 5 years to 65 years old. Such individuals were diagnosed cases of malaria (from all four types Plasmodium species), screened by careful history and physical examination complying with the inclusion criteria. All of the subjects were positive for malarial parasites by direct microscopy.

The method of treating an individual suffering from malaria was performed by a procedure in which the first, second, third and fourth compositions were delivered to the infected individual without identifying the Plasmodium species affecting said individual.

The study used the test drug combination of Artemether (60 mg solution in a Sublingual Nano Spray) as loading dose, Artesunate 50 mg/tablet, Berberine 400 mg/tablet and Primaquine 15 mg/tablet. Directions and dosages for age group and weight were performed based on Table 1

TABLE 9
Dosage Table

| Adult | Adult/Adolescent | Children | Primaquine |
|---|---|---|---|
| (>75 Kg or | (30-75 Kg or 66- | (15-30 Kg or | (0.75 mg |
| 165 lbs.) | 165 lbs) | 33-66 lbs) | base/kg.) |
| Artemether | Artemether | Artemether | Day 4 - single |
| 3 tabs Packet A | 2 tabs Packet A | 1 tab Packet A | dose |
| 2 tabs Packet B | 1 tab Packet B | 1 tab Packet B | P. falciparum |
| for the 1^{st} 12 | for the 1^{st} 12 | for the 1^{st} 12 | P. malariae |
| hrs then repeat | hrs; repeat by | hrs then repeat by | Day 4-17 - |
| the same dose for | taking 1 tab | taking 1 tab | daily for 14 |
| the second 12 hrs | Packet A | Packet A | days |
| Then take | 1 tab Packet B | 1 tab Packet B | P. vivax |
| 2 tabs Packet A | for the second 12 | for the 2^{nd} 12 | P. ovale |
| 2 tabs Packet B | hrs. | hrs then | |
| Every 24 hours | Then take 1 | 1 tab Packet A | |
| for three | tablet from | 1 tab Packet B | |
| additional days. | Packet A & B | every 24 hours | |
| | every 24 hours | for 3 additional | |
| | for 3 additional | days | |
| | days. | | |

Packet A=artesunate
Packet B=berberine

All subjects had initial CBC with Actual Platelet count determined and repeated after four days for uncomplicated and complicated malaria cases. Blood smears for malarial parasites (BSMP) were done daily until the result became negative. Other baseline laboratory examinations like Clotting Time, Bleeding Time, Prothrombin Time (PT), Partial Prothrombin Time (PTT), blood test for aspartate aminotransferase (AST), alanine aminotransferase (ALT), Total Bilirubin, B1, B2, Serum Creatinine, BUN, Random Blood Sugar, Electrocardiogram and Arterial Blood Gas Determination were done upon enrollment and were repeated five days after for complicated cases. All subjects came back for follow up and BSMP were repeated on Day 14, 21 and 28.

Daily assessments of all the subjects were performed. The research subjects were monitored at least 30 days after the last dose of the TriAct therapy. Statistical analyses were done using independent Samples T-test, Chi Square Test for independence using SPSS 11.5.

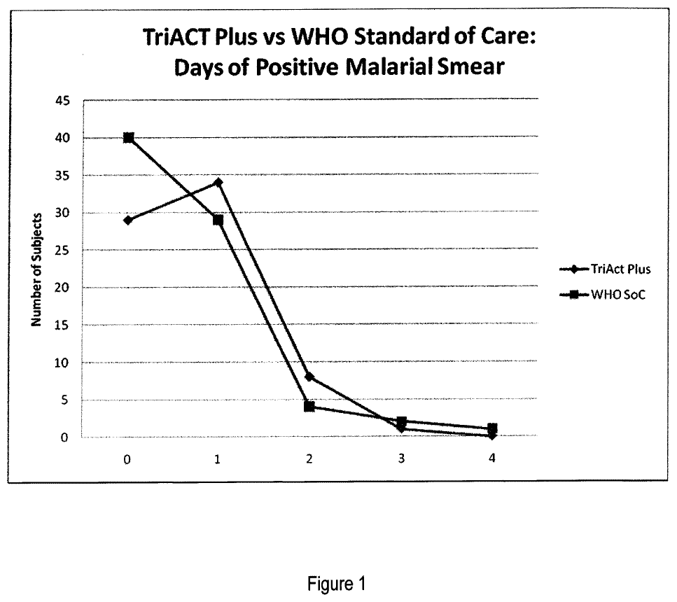

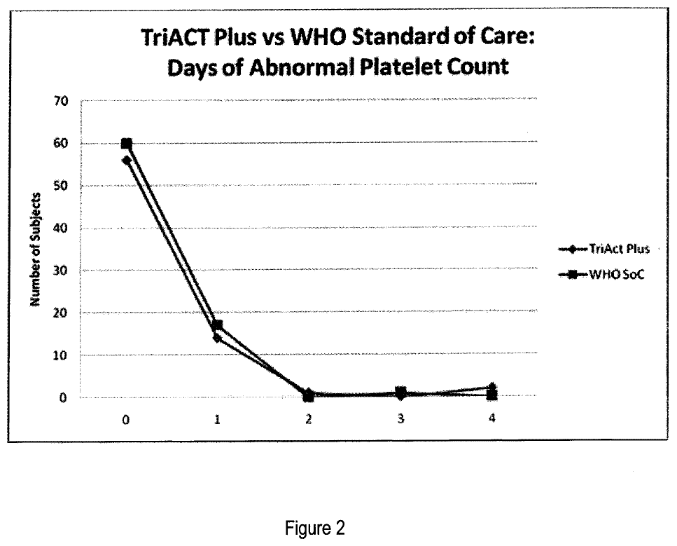

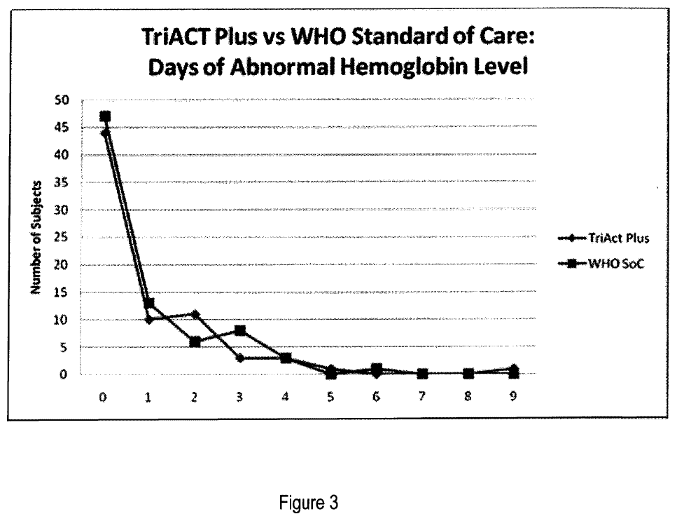

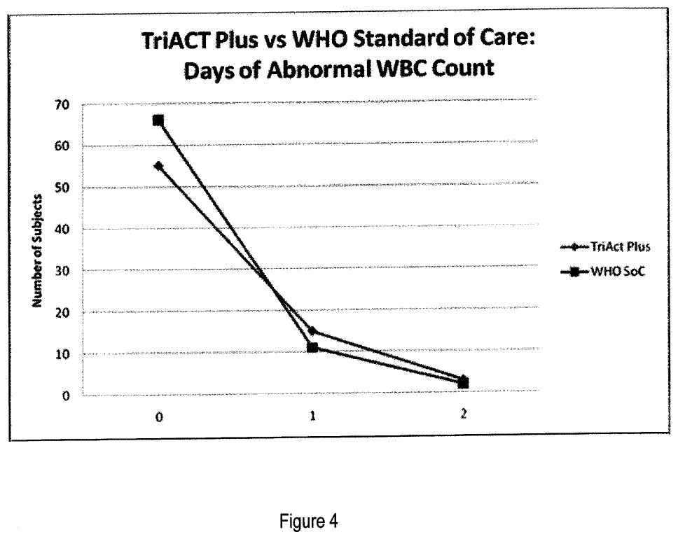

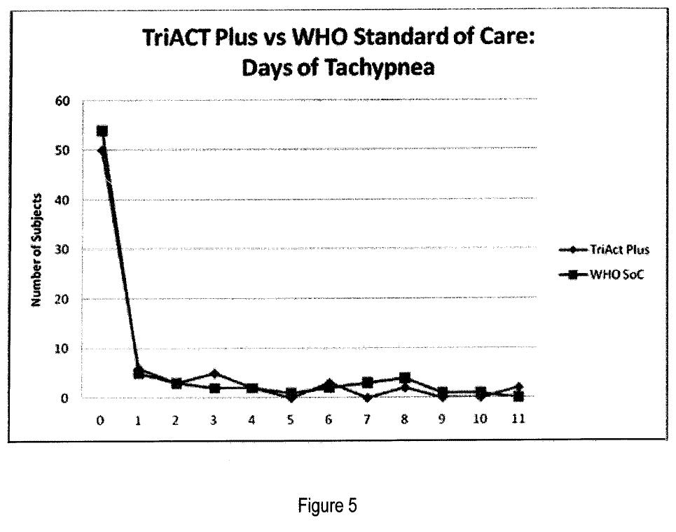

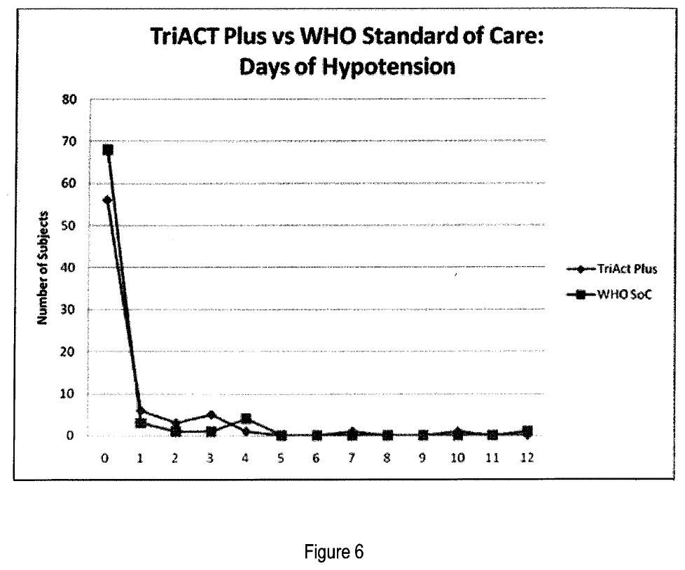

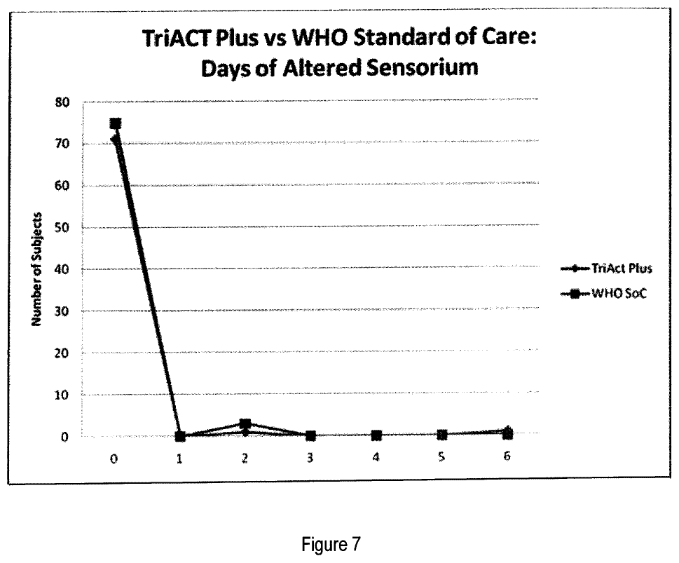

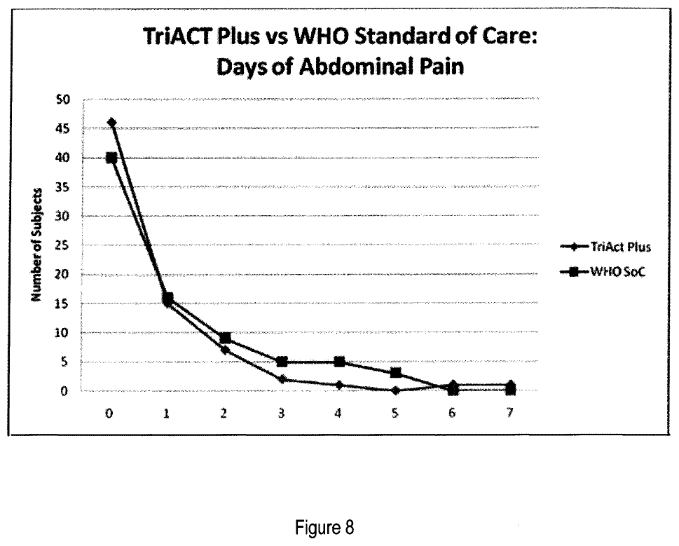

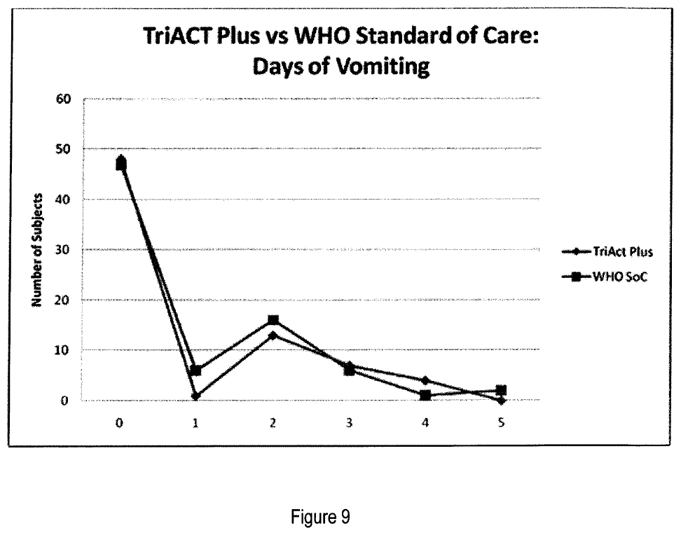

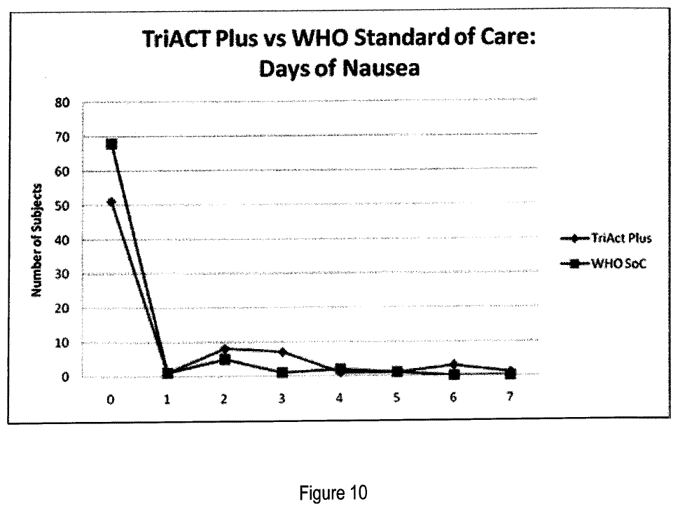

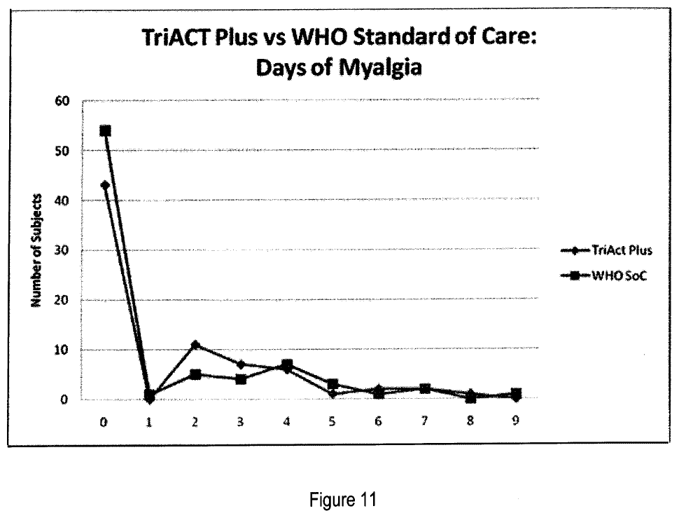

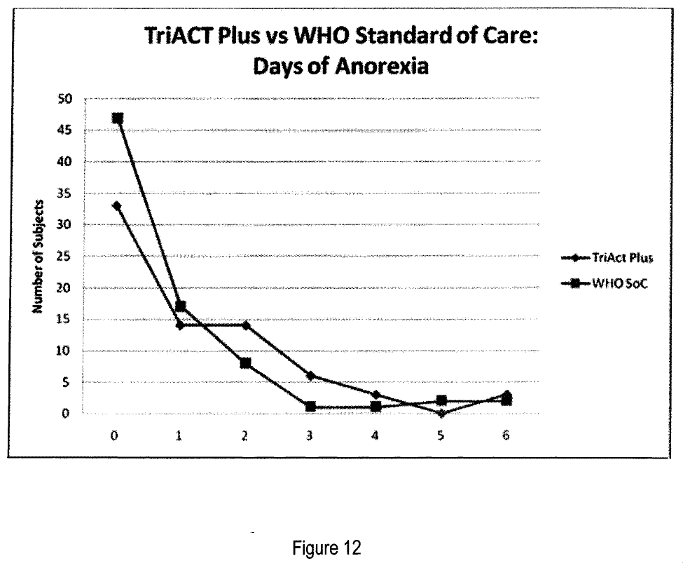

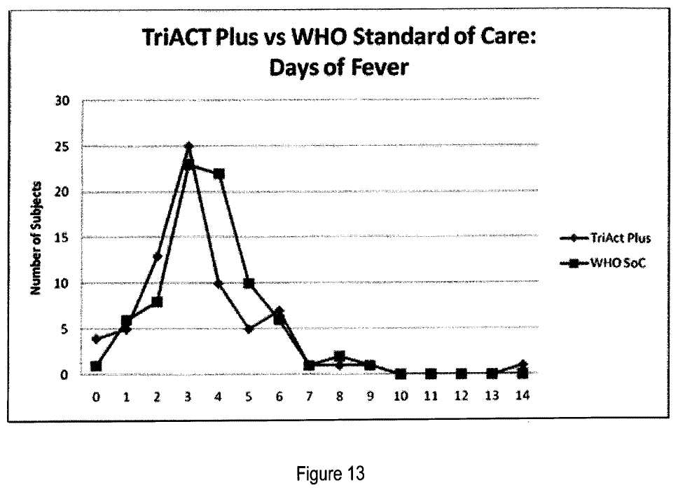

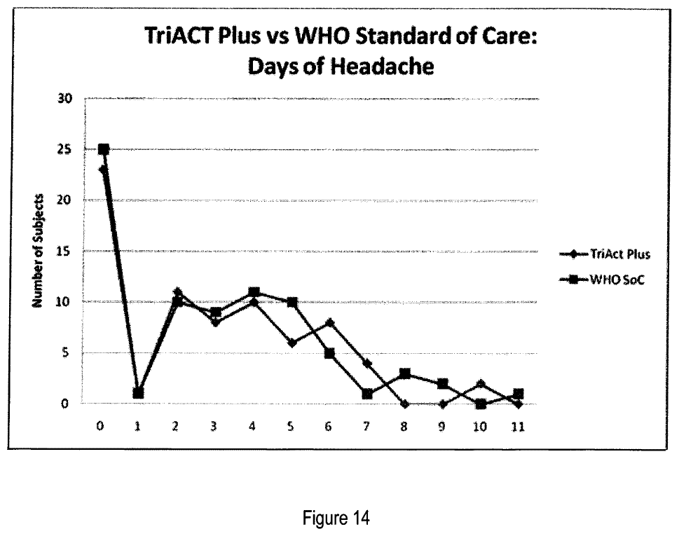

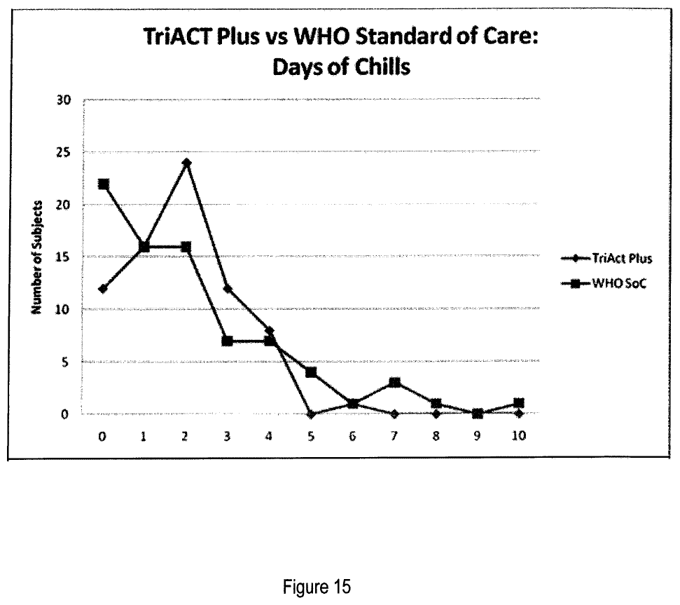

Results: As can be seen by the FIGS. 1-15, the TriAct Plus therapy was show to be comparable to the WHO Standards of Care illustrating that the therapy was effective Current treatment modalities for malaria are effective only if the proper drugs are administered for the specific Plasmodia species infecting the patient. The most common infections are with P. falciparum, P. vivax, P. malariae, P. ovale and mixed infections with P. falciparum and P. vivax. Correct species identification required when using the standard of care is problematic and time consuming. This is especially true in endemic areas where the diagnostic tests are not available. This makes the choice of treatment difficult in situations where immediate treatment is urgently needed. The TriAct Plus therapy is not dependent on proper species identification and can be administered immediately when the first clinical symptoms appear including patients suffering from fever of unknown origin.

In addition, the use of a delivery for artemether via a sublingual (spray) mechanism allows for the treatment of complicated and severe forms of Malaria especially in the absence of parenteral forms. Such form may further serve as a treatment option regardless of the level of consciousness of the patient (cerebral malaria), even when treatment is critical in the most remote areas. Such treatment options are lacking using current stand of care protocols. Applicant's method eliminates the first pass effect (the drug bypasses the liver, thereby reaching the systemic circulation immediately) without sacrificing the serum concentration. Most importantly, the treatment using the TriAct Plus therapy is needle free and can be used anywhere in the world.

Individuals receiving the TriAct Plus therapy did not experience any adverse reactions like hypoglycemia, palpitations and cinchonism which are typically observed with quinine use or its derivatives. Other known side effects using traditional treatments were not observed as well.

The results showed that the TriAct Plus therapy was as effective as standard WHO Standard of Care treatments when species identification (required in standard of care) was correct and the proper drugs were used. The advantage of the TriAct Plus therapy is that such protocol is the only known universal therapy in that it does not require the identification of the specific Plasmodium species which cause the disease. As such, whatever species the individual is infected with, the current TriAct Plus protocol was shown to be effective. This is important in areas where rapid species identification is not available or may come too late, thereby delaying treatment and risking death. It is known that treatment with the current standard of care protocols have many severe side effects in complicated cases, especially with the use of quinine. The TriAct Plus therapy demonstrated no incidence of recurrence, recrudescence, and relapse. Finally, the present protocol resulted in the clearance of gametocytes. None of the subjects came back with any symptoms within a month period.

The method of treatment can be facilitated by the use of a kit including any component as described throughout the specification. In an illustrated embodiment, the method of treatment can be facilitated by the use of a kit comprising an injectable artemether solution as a single dosage unit contained in an ampoule, injection vial, or as an individual single, preloaded injection syringe with needle, a first packet containing artesunate tablets, a second packet containing berberine tablets, and a third packet containing primaquine, or its derivatives or salts, preferably primaquine phosphate. An individual begins the method in accordance with the present invention by administering the loading dose of the artemether. The artemether is delivered to the individual as an injection, via IV or IM route of administration, or preferably by a sublingual spray. The second, third, and fourth compositions are delivered as previously described in Examples 2-5, depending on the weight and age of the individual user.

As described in the above examples, the components of the present invention can be supplied in a kit to aid in delivery and use in areas where distribution channels and/or medical communities are not easily accessible or established. In accordance with the present invention, the kit may include a secure delivery package in the form of a self sealing blister pack, zip standup pouches, foil pouches which include the single use spray bottle and one day, two day, three day, four day, five plus day packs of the artesunate and berberine, and one day and fourteen day packs of primaquine. If injectable artemether is used, needles, syringes, injection bottles, or pre-loaded syringes with capped needles may be included. The kit preferably contains directions and/or any other instructions for the proper use and storage of the components of the kit.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method of treating malaria comprising the steps of:
on day one, administering to an individual suffering from or suspected of suffering from malaria the following:
(1) sublingually, via a nanospray, a single dosage of between 50 mg and 80 mg of artemether or its pharmaceutically acceptable salt, said artemether or its pharmaceutically acceptable salt delivered to an area under said individual's tongue, said single dosage remaining under said individual's tongue for at least 30 seconds;
(2) after between 5 and 30 minutes of administration of said artemether or its pharmaceutically acceptable salt, administering to said individual first dosages of a therapeutically effective amount of artesunate or its pharmaceutically acceptable salts and a therapeutically effective amount of berberine, or its pharmaceutically acceptable salts, each first dosage of said artesunate or its pharmaceutically acceptable salts and said berberine, or its pharmaceutically acceptable salts, are based on said individual's weight or age;
(3) at 12 hours post administration of said first dosages, a second dosage of said artesunate or its pharmaceutically acceptable salts and said berberine, or its pharmaceutically acceptable salts, each second dosage of said artesunate or its pharmaceutically acceptable salts and said berberine, or its pharmaceutically acceptable salts, are based on said individual's weight or age;
on day two, administering to said individual suffering from or suspected of suffering from malaria the following:
(1) a third dosage of therapeutically effective amounts of said artesunate, or its pharmaceutically acceptable salt, and berberine, or its pharmaceutically acceptable salt, said third dosage based on said individual's weight or age;
on day three, administering to said individual suffering from or suspected of suffering from malaria the following:
(1) a fourth dosage of therapeutically effective amounts of said artesunate, or its pharmaceutically acceptable salt, and berberine, or its pharmaceutically acceptable salt, said fourth dosage based on said individual's weight or age;
on day four, administering to said individual suffering from or suspected of suffering from malaria, the following:
(1) a fifth dosage of therapeutically effective amounts of said artesunate, or its pharmaceutically acceptable salt, and berberine, or its pharmaceutically acceptable salt, said fifth dosage based on said individual's weight or age, and
(2) a first dosage of a therapeutically effective amount of primaquine, or its pharmaceutically acceptable salts; said first dosage of said primaquine based on said individual's weight or age; and
after day four, administering to said individual suffering from or suspected of suffering from malaria, the following:
(1) a therapeutically effective amount of primaquine, or its pharmaceutically acceptable salts, daily for a period of between one to fourteen days.
wherein, all forms of parasites Plasmodium falciparum, Plasmodium vivax, Plasmodium ovale, Plasmodium malariae, and Plasmodium knowlesi are eliminated within two (2) days of beginning treatment, incidence of recurrence, recrudescence, or relapse is prevented, and adverse reactions associated with primaquine, or its pharmaceutically acceptable salts are eliminated.

2. The method of treating malaria according to claim 1 wherein said sublingual spray comprises a mucosal absorption enhancer.

3. The method of treating malaria according to claim 1 wherein said artesunate, or its pharmaceutically acceptable salt, said berberine, or its pharmaceutically acceptable salt, and said primaquine, or its pharmaceutically acceptable salts are delivered via an oral route.

4. The method of treating malaria according to claim 1 wherein said artesunate, or its pharmaceutically acceptable salt, said berberine, or its pharmaceutically acceptable salt, and said primaquine, or its pharmaceutically acceptable salts are in the form of a pill, tablet, or capsule.

5. The method of treating malaria according to claim 1, wherein said artesunate, or its pharmaceutically acceptable salt, said berberine, or its pharmaceutically acceptable salt, and said primaquine, or its pharmaceutically acceptable salts are in the form of an injectable liquid.

6. The method of treating malaria according to claim 1, wherein said primaquine salt is primaquine phosphate.

7. The method of treating malaria according to claim 1, wherein said artesunate, or its pharmaceutically acceptable salt, said berberine, or its pharmaceutically acceptable salt, and said primaquine, or its pharmaceutically acceptable salts include a pharmaceutically acceptable excipient.

8. The method of treating malaria according to claim 1, wherein said artesunate, or its pharmaceutically acceptable salt, said berberine, or its pharmaceutically acceptable salt, and said primaquine, or its pharmaceutically acceptable salts include a pharmaceutically acceptable carrier.

9. A kit for administering a plurality of compositions to an individual suffering from or suspected of suffering from malaria comprising:
a first composition, said first composition comprising a therapeutically effective amount of an artemisinin derivate;
a second composition, said second composition comprising a therapeutically effective amount of a second artemisinin derivate, said second artemisinin derivate differing from said first composition;
a third composition comprising an effective amount of berberine, or its pharmaceutically acceptable derivatives; and
a fourth composition comprising an effective amount of primaquine, or its pharmaceutically acceptable derivatives or salts.

10. The kit according to claim 9 wherein said first composition is artemether and said second composition is artesunate.

11. The kit according to claim 10 wherein said artemether is a spray.

12. The kit according to claim 9 wherein said second and said third composition is in an injectable form.

13. The kit according to claim 12 wherein said second, said third, and said four compositions are in the form of a pill, tablet, or capsule.

14. The kit according to claim 9 wherein said second, said third, and said fourth compositions are in pre-packed dosages.
